(19) United States
(12) Patent Application Publication
(10) Pub. No.: US 2013/0183369 A1
(43) Pub. Date: Jul. 18, 2013

(54) Means of inducing cellular exocytosis and uses thereof

(71) Applicant: John Douglas Furber, Gainesville, FL (US)

(72) Inventor: John Douglas Furber, Gainesville, FL (US)

(21) Appl. No.: 13/548,006

(22) Filed: Jul. 12, 2012

Related U.S. Application Data
(60) Provisional application No. 61/507,013, filed on Jul. 12, 2011.

(51) Int. Cl. A61K 35/68; C12N 13/00; A61K 45/00

(52) U.S. Cl. CPC A61K 35/68; A61K 45/00; C12N 13/00
USPC 424/450; 435/375; 435/173.4; 424/780; 514/58; 514/1.1; 424/141.1

(57) ABSTRACT

Means, compositions, and uses for triggering exocytosis of lysosomes in living cells. During exocytosis, lysosomes travel to the plasma membrane of the cell and dump their contents outside of the cells, thus removing accumulations of harmful, reactive metabolic by-products, such as lipofuscin. Consequently, cells function better, thus improving their vitality and the vitality of people, animals, and cell cultures. Degenerative diseases can be prevented or reversed. Embodied methods include electrical pulses, sonic vibrations, mechanical pressure, and mixtures of substances, which may include drugs, proteins, antibodies, or liposomes, which can be combined, injected, or administered transdermally or orally. Other embodiments are described and shown.

### CROSS-REFERENCE TO RELATED APPLICATIONS

This application claims the benefit of provisional patent application Ser. No. 61/507,013, filed 2011 Jul. 12 by the present inventor.

### FEDERALLY SPONSORED RESEARCH OR DEVELOPMENT

Not applicable.

### SEQUENCE LISTING

Not applicable.

### BACKGROUND OF THE INVENTION

### Prior Art

The following is a tabulation of some prior art that presently appears relevant:

U.S. Patents

| Pat. No. | Kind Code | Issue Date | Patentee |
|---|---|---|---|
| 6,174,890 | B1 | 2001 Jan 16 | Riga |

U.S. Patent Application Publications

| Publication Nr. | Kind Code | Publ. Date | Applicant |
|---|---|---|---|
| 20110046377 | A1 | 2011-02-24 | Schraermeyer |

Foreign Patent Documents

| Foreign Doc. | | | | |
|---|---|---|---|---|
| Nr. | Cntry Code | Kind Code | Pub. Date | App or Patentee |
| EP2080513 | EC | A1 | 2008 Jun 12 | Schraermeyer |

### NONPATENT LITERATURE DOCUMENTS

Alberts et al. Molecular Biology of the Cell. (5th Edition, Garland Science, 2008).
Benavides S H, Monserrat A J, Farina S, Porta E A, Sequential histochemical studies of neuronal lipofuscin in human cerebral cortex from the first to the ninth decade of life. Arch. Gerontol. Geriatr. 34, 219-231 (2002).
Brody H, The deposition of aging pigment in the human cerebral cortex. J. Gerontol. 15, 258-261 (1960).
Brunk U T, Terman A. Lipofuscin: Mechanisms of Age-related Accumulation and Influence on Cell Function. Free Radical Biology & Medicine 2002; 33(5):611-619.
Burleigh and Andrews. Current Opinion in Microbiology. “Signaling and host cell invasion by Trypanosoma cruzi”, (1998 1:461-465)
Corrotte M, Fernandes M C, Tam C, Andrews N W. Traffic. “Toxin pores endocytosed during plasma membrane repair traffic into the lumen of MVBs for degradation”. (2012 March; 13(3):483-494).
Cuervo A M, Bergamini E, Brunk U T, Dröge W, French M, Terman A. Autophagy and aging: the importance of maintaining “clean” cells. Autophagy. 2005 October-December; 1(3):131-40. Epub 2005 Oct. 13. Review. PubMed PMID: 16874025.
de Grey A D, Alvarez P J, Brady R O, Cuervo A M, Jerome W G, McCarty P L, Nixon R A, Rittmann B E, Sparrow J R. Ageing Res Rev. “Medical bioremediation: prospects for the application of microbial catabolic diversity to aging and several major age-related diseases”. (2005 August; 4(3):315-38.)
Fernandes M C, Cortez M, Flannery A R, Tam C, Mortara R A, Andrews N W. J Exp Med. “Trypanosoma cruzi subverts the sphingomyelinase-mediated plasma membrane repair pathway for cell invasion.” (2011 May 9; 208(5):909-921).
Medina D L, Fraldi A, Bouche V, Annunziata F, Mansueto G, Spampanato C, Puri C, Pignata A, Martina J A, Sardiello M, Palmieri M, Polishchuk R, Puertollano R, Ballabio A. Transcriptional activation of lysosomal exocytosis promotes cellular clearance. Dev Cell. 2011 Sep. 13; 21(3):421-430.
Riga et al. Brain Research. “Effects of Centrophenoxine on the Lipofuscin Pigments in the Nervous Systems of Old Rats”, (72, 1974:265-275)
Settembre C, Fraldi A, Jahreiss L, Spampanato C, Venturi C, Medina D, de Pablo R, Tacchetti C, Rubinsztein D C, Ballabio A. (2008). A block of autophagy in lysosomal storage disorders. Hum. Mol. Genet. 17, 119-129.
Terman A, Brunk U T. Lipofuscin: mechanisms of formation and increase with age. APMIS 106:265-276; 1998.
Terman et al. Antioxidants & Redox Signaling. “Mitochondrial Turnover and Aging of Long-Lived Postmitotic Cells: The Mitochondrial-Lysosomal Axis Theory of Aging.” (Liebert, 12(4), 2010.)

Human aging proceeds by several chains of events, but most of them result because cellular repair processes do not keep up with accumulating cellular damage. Young cells are able to repair and rejuvenate themselves, in part, because they have an active recycling system. A membrane within the cell (an autophagosome) engulfs worn-out structures and then is transported to a tiny bag called a lysosome. This process is called autophagy. Inside the lysosome, hydrolytic enzymes dissolve and break down worn-out cell structures into their components in order to allow the cell to rebuild new structures, using the recycled pieces. Repair slows down with age, especially in heart muscle cells and brain neurons, which live for a human lifetime, but never divide.

For more than 100 years, observations by medical scientists have reported that excess materials accumulate gradually over time in the cells of aging humans and animals. Excess materials are especially noticeable in the lysosomes of non-dividing cells, such as heart muscle or neurons. (Terman 2010) Over the years, small amounts of indigestible material build up inside lysosomes and bond together to form a plastic-like polymer called lipofuscin, which interferes with lysosome function. Lipofuscin accumulates over time (Brunk; Termam 1998; Benavides; Brody) because iron-catalyzed chemical reactions attach more molecules to the growing mass of lipofuscin in the lysosome. Recycling slows down as working space inside the lysosome is crowded out by the growing mass of lipofuscin. Hydrolytic enzymes are less available when many are transported futilely to nonproductive lysosomes; the few remaining functional lysosomes do not receive enough enzymes to effectively break down all of the materials that need to be recycled (Brunk; Cuervo 2005). With less lysosomal digestion, the lysosomes can no longer receive as many cargos from the autophagosomes, so autophagy slows down (Settembre; Terman 2010). Cell structures are not replaced as often. Cell membranes therefore accumulate increasing numbers of oxidized lipids, and the cells fill up with accumulating worn-out mitochondria and damaged proteins. As cell repair slows down, the cells no longer function well. Furthermore, increasing volumes of space within the cells are taken up by the lipofuscin-clogged lysosomes (Terman 1998; Brunk), further hindering the ability of the cells to perform their functions.

Lysosomes are well known as tiny membrane-bounded vesicles, which exist inside of living cells. (Alberts) These accumulations of excess materials have been generally regarded as by-products of metabolism, and are often referred to as “lipofuscin” or “ceroid”. In the following discussion, I may refer to all such excess materials as “lipofuscin”, but with the understanding that the discussion is meant to include the full range of metabolic by-products that may accumulate in lysosomes over the passing of time.

Lipofuscin accumulation is especially harmful in cells that work hard, consume a lot of oxygen, and live a very long time without dividing, such as heart muscle, skeletal muscle, neurons in the brain, and retinal pigment epithelial (RPE) cells in the eye. Sometimes this kills the cell. Other cells stay alive, but do not function well [Brunk; Terman 2010].

Lipofuscin accumulation also causes problems in fibroblasts, stem cells, and progenitor cells. These are cells that occasionally divide for special purposes, but spend long periods of time without dividing. Lipofuscin can actually prevent these cells from dividing when they are asked to, such as when fibroblasts need to repair a wound, or when stem cells and progenitors need to produce replacement cells.

There is substantial evidence that lipofuscin accumulation interferes with cell functions and can result in the deaths of cells. (Terman 2010.) This can cause or contribute to some of the serious, deadly, or debilitative diseases of old age. Extreme examples of clogged lysosomes are seen in rare genetic disorders called “lysosomal storage diseases”, which can be deadly even for young children. More commonly, lysosomes clogged with lipofuscin are seen in heart muscle cells and neurons of old people and old animals (Terman 2010).

For example, lipofuscin-loaded lysosomes can occupy a significant volume inside cells, and thereby physically obstruct vital cellular functions. Furthermore, some compositions of lipofuscin are chemically highly reactive. They generate free radicals and contribute to pathogenic chemical reactions inside the cell in which they are located.

In prior years, there have been proposals that the rate of accumulation of lipofuscin might be reduced by various proposed treatments, such as oral consumption of centrophenoxine. (Riga 1974) However, until now, no such proposed treatments have been generally accepted by the scientific or medical communities as being able to slow the rate of accumulation of lipofuscin.

Aubrey de Grey proposed a concept whereby enzymes capable of digesting lipofuscin would be sought in soil microbes, isolated, identified, manufactured, and delivered to cells in the human body so that the cells could better digest their own accumulated lipofuscin. (de Grey) However, that conceptual proposal has not yet been reduced to practice.

Recently, Schraermeyer applied for a US patent (Pat Appl #US 2011/0046377) on medications for use in the treatment of age-related macular degeneration (AMD) and dry AMD. Schraermeyer's discussion in that patent application mentions that lipofuscin accumulation in RPE cells of the eyes is associated with the pathology of AMD, but the claims in his patent application do not mention either lipofuscin or exocytosis. The uses claimed by Schraermeyer are based upon specific chemical structures, known as tetrahydropyridoethers, which his discussion says would remove lipofuscin from RPE cells in the eyes. However, his application does not claim any uses other than for treatment of AMD or dry AMD.

In recent years, medical scientists have been discovering steps in natural mechanisms by which a living cell can transport materials from the inside of said cell to the outside of said cell. In some cases, said transport occurs by a natural process referred to as “exocytosis” in which vesicles containing materials move from the interior of said cell's cytoplasm to the bounding membrane of the cell (the plasma membrane). (Burleigh) Then the vesicle membrane fuses with the plasma membrane as a hole forms in the plasma membrane, while the vesicle turns inside-out, dumping its contents outside the cell. (Alberts) Exocytosis is seen in several natural cellular activities, including neural synaptic vesicle release, and in the secretion of cellular products, such as insulin. Exocytosis has also been reported to provide membrane material from vesicle membranes to patch holes in the plasma membrane. During some disease conditions, such as when cells are attacked by particular parasites or microbial toxins, observers have reported that exocytosis is stimulated to occur, and that said exocytotic vesicles are actually lysosomes. However, these observations were always made in young cells, which did not contain any lipofuscin. [Andrews] A particularly well-studied parasite that induces exocytosis is Trypanosoma cruzi, which triggers an exocytotic pathway when it makes contact with the outer membrane of a human cell. A microbial toxin that can trigger an exocytotic pathway is streptolysin-O, which embeds in the plasma membrane of a cell, and forms a channel through which calcium ions can leak into the cell.

Some of the mechanistic sequences or pathways of cellular events by which natural exocytosis takes place utilize signals of calcium ions or activation of the TFEB gene. (Medina) However, the analysis and description of said pathways has previously been reported only in young cells, which did not contain any lipofuscin.

It has been reported that pore-forming compounds, such as streptolysin-O, can become embedded in cellular membranes, temporarily increasing their permeability to hydrophilic ions and molecules, such as calcium ions. (Carrotte)

The Andrews lab has reported that some cells will naturally exocytose lysosomes in response to a hole in the plasma membrane. (Fernandes) The lysosomal exocytosis contributes lysosomal membrane materials to patch the hole in the plasma membrane. However, their observations did not report on any cells that contained lipofuscin.

### ADVANTAGES

Accordingly, several advantages of one or more aspects are as follows: Removing lipofuscin will restore effective functioning of lysosomes, so that improved repair will rejuvenate aging cells to a younger condition. Therapeutically induced exocytosis of lipofuscin-loaded lysosomes improves the health of cells, tissues, organs, people, and animals. With a smaller burden of lipofuscin-loaded lysosomes, cells will function better, and reactive chemical damage to intracellular molecules would be reduced. With healthier cells, the tissues, organs, and physiology of humans and animals will be more resistant to debilitative and degenerative diseases. By reducing the quantity of lipofuscin in old or diseased cells, they can be restored to a healthier condition, and consequent disease conditions can be prevented, reduced, or restored to health.

### BRIEF SUMMARY OF THE INVENTION

Embodiments of the claimed invention cover methods, uses, and compositions of matter that trigger biological signaling pathways in living cells, which result in the cells secreting excess materials, including lipofuscin, by exocytosis of their lysosomes. Several embodiments of this invention induce exocytosis by several methods. Embodiments of the claimed invention cover the use of exocytosis induction for the treatment of diseases and for the removal of materials from inside cells in order to improve vitality and prevent or treat diseases in people, animals, and cells.

### DRAWINGS

Drawings—Figures: Not applicable.
Reference Numerals—Not applicable.

### DETAILED DESCRIPTION OF THE INVENTION

### Detailed Description

### First Embodiment

In this embodiment, chemical therapeutic substances are delivered to living cells. These substances are composed of a combination of one or more of the following substances: gp82 from Trypanosome cruzi, one or more calcium channel agonists, one or more pore-forming substances, such as streptolysin-O, which embed in cellular membranes, one or more compounds in the family of tetrahydropyridoethers, one or more compounds in the family of cyclodextrine compounds, and materials to enhance distribution and delivery of said substances to membranes of living cells. These substances are delivered to cells at a concentration sufficient to trigger an exocytotic pathway, but at a concentration low enough to avoid killing most cells.

### Operation

### First Embodiment

Chemical therapeutic substances trigger a biochemical sequence of events in living cells, resulting in exocytosis of lysosomes. For effective localized delivery, numerous low dosage injections are made within and close to the tissues being treated. After a period of observation, additional treatments can be made, with adjustments to dosages and sites of injection. Lysosomes are stimulated to move out to the plasma membranes of their cells. The lysosomal membrane fuses with the plasma membrane, forming an opening in the plasma membrane. The lipofuscin contained in the exocytosed lysosomes is released to the exterior of the cell. Consequently, the cells are healthier when then are no longer carrying lipofuscin-loaded lysosomes inside them.

### Description

### Additional Embodiment

In this embodiment, brief electrical pulses are sent to the cell. They are at an intensity sufficient to trigger an exocytotic pathway, but at an intensity low enough to avoid killing most cells.

### Operation

### Additional Embodiment

Electrical pulses can cause small temporary openings in the plasma membrane of a cell. The cell responds by repairing the openings. During repair, the cell delivers lysosomes to the site of the opening in the plasma membrane, and exocytoses them so that they provide lysosomal membrane material to patch the hole in the plasma membrane. For effective localized treatment, numerous energy pulses are made within and close to the tissues being treated. After a period of observation, additional treatments can be made, with adjustments to energy levels and sites of the electrodes.

### Description

### Additional Embodiment

In this embodiment, mechanical pressure or sonic vibrations are delivered to the cell. They are at an intensity sufficient to trigger an exocytotic pathway, but at an intensity low enough to avoid killing most cells.

### Operation

### Additional Embodiment

Mechanical pressure or sonic vibrations can cause small temporary openings in the plasma membrane of a cell. The cell responds by repairing the openings. During repair, the cell delivers lysosomes to the site of the opening, and exocytoses them so that they provide lysosomal membrane material to patch the hole in the plasma membrane. For effective localized treatment, pulses of mechanical or sonic energy are made within and close to the tissues being treated. After a period of observation, additional treatments can be made, with adjustments to energy levels and sites of the transducers.

### Description

### Additional Embodiment

In this embodiment, chemicals that can induce exocytosis are harvested and purified from cell cultures of Trypanosoma cruzi parasites.

### Operation

### Additional Embodiment

In this embodiment, a batch of Trypanosoma cruzi parasites is grown by commonly employed cell culture methods. The Trypanosoma cruzi cells are killed to render them non-infective. Then they are macerated, and the contents separated by commonly employed biochemistry techniques. The various biochemical fractions are assayed by applying them to cultures of heart muscle cells that have lysosomes filled with waste. The responses of the cells are observed with a fluorescence confocal microscope, which can easily see movement of lipofuscin because it is autofluorescent. Active fractions, which are able to induce the heart cells to expel their wastes by exocytosis, are formulated into therapeutic medicaments or dietary supplements, which may be administered orally, transdermally, or by injection. For effective localized delivery, numerous low dosage injections are made within and close to the tissues being treated. After a period of observation, additional treatments can be made, with adjustments to dosages and sites of injection.

### Description

### Additional Embodiment

In this embodiment, chemicals that can induce exocytosis are synthesized or cultured and purified after determining the chemical structure of chemical triggers of exocytosis discovered by analyzing components from cell cultures of Trypanosoma cruzi parasites.

### Operation

### Additional Embodiment

Another embodiment of this invention synthesizes active molecules by determining the structure and sequence of the molecules in the active fraction of macerated Trypanosoma cruzi parasites. Structure and sequence determination of proteins and glycoproteins uses well-established techniques of biochemistry and biophysics. The sequence and structure data is used to synthesize peptides and glycopeptides of similar sequence and structure. Well-known techniques of biochemistry are used to synthesize peptides and glycopeptides. The synthetic peptides and glycopeptides are assayed by applying them to cultures of heart muscle cells that have lysosomes filled with waste. The responses of the cells are observed with a fluorescence confocal microscope, which can easily see movement of lipofuscin because it is autofluorescent. The active peptides and glycopeptides, which are able to induce the heart cells to expel their wastes by exocytosis, are formulated into injectable, oral, or transdermal therapeutic medicaments or dietary supplements. For effective localized delivery, numerous low dosage injections are made within and close to the tissues being treated. After a period of observation, additional treatments can be made, with adjustments to dosages and sites of injection.

### Description

### Additional Embodiment

Another embodiment of this invention uses phage display libraries to find proteins and monoclonal antibodies that can trigger exocytosis.

### Operation

### Additional Embodiment

Well-established techniques can generate phage display libraries that can be tested by applying them to cell cultures and screening for stimulation of exocytosis. For example, screening could be done on heart muscle cells that have lysosomes filled with waste. The pages displaying active proteins or antibodies, which are able to induce the heart cells to expel their wastes by exocytosis, are then produced in large quantities by techniques well know in the biotechnology industry. The active proteins or antibodies are formulated into injectable, oral, or transdermal therapeutic medicaments or dietary supplements. For effective localized delivery, numerous low dosage injections are made within and close to the tissues being treated. After a period of observation, additional treatments can be made, with adjustments to dosages and sites of injection.

### CONCLUSION, RAMIFICATIONS, AND SCOPE

Uses include, but are not limited to: treating lysosomal storage diseases, or for prevention or treatment of neurodegeneration, Alzheimer's disease, dementia, senility, cardiac myopathy, heart disease, macular degeneration, sarcopenia, and degenerative conditions associated with advanced age in people, animals, and cell cultures. Further uses include maintaining and restoring the health and fitness of people and aging pets, livestock, zoo animals, endangered species, and other animals. Other uses include research reagents for inducing exocytosis and lysosomal movement in lipofuscin-loaded cell cultures, tissue cultures, and research animals.

Embodiments of the invention may be injected, rubbed onto the skin, or administered orally to people or animals or cultured cells, tissues, or organs. Injections may be into the skin, blood, brain, heart muscle, eye, retina, or cerebrospinal fluid. Repeated treatments and localized treatments can be performed to treat specific conditions. Any of several methods of inducing lysosomal exocytosis may be used, alone or in combination, including chemical, electrical, or sonic stimulation. Other embodiments of this invention are indicated by the observation that when small holes are made in the plasma membranes of living cells, the cell induces signaling pathways of lysosomal exocytosis to patch the holes, which results in the cells secreting waste products by exocytosis of their lysosomes. Holes can be produced by mechanical forces, sonic vibrations, electrical stimulations, or by chemical treatments.

Although the description above contains many specificities, these should not be construed as limiting the scope of the embodiments but as merely providing illustrations of some of several embodiments. For example, many different combinations of substances will trigger biochemical pathways resulting in exocytosis of lysosomes. Various molecular structures, such as liposomes and antibodies, can be used to deliver exocytosis-triggering substances to tissues and cells in appropriate locations and amounts. Thus the scope of the embodiments should be determined by the appended claims and their legal equivalents, rather than by the examples given.

I claim:

1. methods of triggering biological signaling pathways that cause exocytosis by lysosomes in living cells.

2. methods of claim 1 using electrical stimulation to trigger said signaling pathways.

3. methods of claim 1 using mechanical pressure or sonic vibrations to trigger said signaling pathways.

4. methods of claim 1 using chemical substances to trigger said signaling pathways.

5. methods of claim 4 in which said chemical substances are composed of a combination of one or more of the following substances: gp82 from Trypanosome cruzi, one or more calcium channel agonists, one or more pore-forming substances, such as streptolysin-O, which embed in cellular membranes, one or more compounds in the family of tetrahydropyridoethers, one or more compounds in the family of cyclodextrine compounds, liposomes, and materials to enhance distribution and delivery of said substances to membranes of living cells.

6. methods of claim 4 in which said chemical substances trigger the same signaling pathway as triggered by the parasite, Trypanosoma cruzi, when it contacts a cell to invade it.

7. methods of claim 4 in which said chemical substances are harvested from parasites, such as Trypanosoma cruzi, and purified.

8. methods of claim 4 in which said chemical substances are designed with a molecular shape or structure similar to the molecules used by the parasite, Trypanosoma cruzi, when it contacts a cell to invade it.

9. methods of claim 4 in which said chemical substances are proteins or monoclonal antibodies found by screening phage-display libraries on cells to assay for exocytosis stimulation.

10. methods of claim 4 in which said chemical substances are delivered orally or transdermally or by injection into the skin, blood, brain, heart muscle, eye, retina, or cerebrospinal fluid.

11. compositions of matter composed of a combination of one or more of the following substances: gp82 from Trypanosome cruzi, one or more calcium channel agonists, one or more pore-forming substances, such as streptolysin-O, which embed in cellular membranes, one or more compounds in the family of tetrahydropyridoethers, one or more compounds in the family of cyclodextrine compounds, liposomes, and materials to enhance distribution and delivery of said substances to membranes of living cells.

12. compositions of matter in which molecular structures are harvested from parasites, such as Trypanosoma cruzi, purified, and formulated into medicaments or dietary supplements that are able to trigger a signaling pathway of exocytosis.

13. compositions of matter in which molecules are designed with a shape or structure similar to molecules in parasites, such as Trypanosoma cruzi, that signal a cell to exocytose its lysosomes when the parasite contacts a cell.

14. compositions of matter, composed of chemicals identified by phage display library screening, which are able to trigger a signaling pathway of exocytosis.

15. uses of the methods of claim 1 for maintaining, enhancing, or restoring health and fitness, or for prevention or treatment of neurodegeneration, Alzheimer's disease, dementia, senility, cardiac myopathy, heart disease, macular degeneration, sarcopenia, degenerative conditions, or lysosomal storage diseases in people, companion animals, livestock, zoo animals, research animals, and cell cultures.
